(19) United States
(12) Patent Application Publication
(10) Pub. No.: US 2019/0133555 A1
(43) Pub. Date: May 9, 2019

(54) ULTRASONIC TRANSDUCER MODULE AND ULTRASONIC ENDOSCOPE

(71) Applicant: Olympus Corporation, Tokyo (JP)

(72) Inventor: Tomoyuki Hatakeyama, Tokyo (JP)

(73) Assignee: Olympus Corporation, Tokyo (JP)

(21) Appl. No.: 16/190,252

(22) Filed: Nov. 14, 2018

Related U.S. Application Data
(63) Continuation of application No. PCT/JP2017/017917, filed on May 11, 2017.

(30) Foreign Application Priority Data
May 20, 2016 (JP) 2016-101628

(51) Int. Cl. A61B 8/00; A61B 8/12

(52) U.S. Cl. CPC A61B 8/4483; A61B 8/14; A61B 8/445; A61B 8/12

(57) ABSTRACT

An ultrasonic transducer module including: piezoelectric elements, each being aligned in the same direction that is a longitudinal direction thereof; electrodes that are formed on surfaces of the respective piezoelectric elements; a substrate that includes wiring members extending from at least one surface of the substrate, the wiring members being connected one by one to the electrodes of the piezoelectric elements; and reinforcing layers that are provided on a surface of each of the wiring members, the surface being opposite to a surface of the corresponding wiring member where the corresponding electrode is connected.

### CROSS-REFERENCE TO RELATED APPLICATION

This application is a continuation of PCT International Application No. PCT/JP2017/017917 filed on May 11, 2017 which claims the benefit of priority from Japanese Patent Application No. 2016-101628, filed on May 20, 2016, the entire contents of which are incorporated herein by reference.

### BACKGROUND

The present disclosure relates to an ultrasonic transducer module and an ultrasonic endoscope that includes an ultrasonic transducer at a distal end of an insertion portion.

Ultrasonic waves can be used to observe characteristics of a tissue of a living body or a material that is an object to be observed. Specifically, an ultrasonic observation apparatus subjects an ultrasonic echo received from an ultrasonic transducer that transmits and receives ultrasonic waves to a predetermined signal process, and thereby obtains information relating to characteristics of an object to be observed.

The ultrasonic transducer includes plural piezoelectric elements that converts an electrical pulse signal into an ultrasonic pulse (acoustic pulse) to irradiate to an object to be observed, and that converts an ultrasonic echo reflected by the object to be observed into an electrical echo signal to output. For example, the piezoelectric elements are aligned along a predetermined direction, and devices involved in transmission and reception are switched thereamong, thereby acquiring ultrasonic echo from the object to be observed.

As types of ultrasonic transducers, various types with which transmission/reception directions of ultrasonic beams differ, such as a convex type, a linear type, and a radial type, have been known. Out of these, in a convex ultrasonic transducer, plural piezoelectric elements are aligned along a curved surface, and each emits an ultrasonic beam toward a diameter direction of the curved surface (for example, refer to Japanese Patent No. 2555376). In Japanese Patent No. 2555376, after arranging plural piezoelectric elements on a flat surface and connecting flexible printed circuits (FPC), the plural piezoelectric elements are bent, thereby manufacturing a convex ultrasonic transducer.

### SUMMARY OF THE DISCLOSURE

An ultrasonic transducer module according to one aspect of the present disclosure includes: piezoelectric elements, each being aligned in the same direction that is a longitudinal direction thereof; electrodes that are formed on surfaces of the respective piezoelectric elements; a substrate that includes wiring members extending from at least one surface of the substrate, the wiring members being connected one by one to the electrodes of the piezoelectric elements; and reinforcing layers that are provided on a surface of each of the wiring members, the surface being opposite to a surface of the corresponding wiring member where the corresponding electrode is connected.

An ultrasonic endoscope according to one aspect of the present disclosure includes: an ultrasonic transducer module that is provided at a distal end portion of the ultrasonic endoscope; and an insertion portion that is inserted into a subject.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically showing an endoscope system according to a first embodiment;

FIG. 2 is a perspective view schematically showing a distal end structure of an insertion portion of an ultrasonic endoscope according to the first embodiment;

FIG. 3 is a perspective view schematically showing a structure of an ultrasonic transducer module according to the first embodiment;

FIG. 4 is a diagram for explaining a structure of a principal part of the ultrasonic transducer module shown in FIG. 3;

FIG. 5 is a schematic diagram showing a structure of a principal part of the ultrasonic transducer module according to the first embodiment, and is a schematic diagram for explaining a structure of a relay board;

FIG. 6 is a partial cross-section for explaining a structure of principal part of the ultrasonic transducer module shown in FIG. 3;

FIG. 7 is a partial cross-section for explaining a structure of a principal part of an ultrasonic transducer module according to a first modification of the first embodiment;

FIG. 8 is a partial cross-section for explaining a structure of a principal part of an ultrasonic transducer module according to a second modification of the first embodiment;

FIG. 9 is a partial cross-section for explaining a principal part of an ultrasonic transducer module according to a third modification of the first embodiment;

FIG. 10 is a schematic diagram showing a structure of a principal part of an ultrasonic transducer module according to a second embodiment, and is a schematic diagram for explaining a structure of a relay board;

FIG. 11 is a partial cross-section for explaining a structure of a principal part of the ultrasonic transducer module according to the second embodiment;

FIG. 12 is a partial cross-section for explaining a structure of a principal part of an ultrasonic transducer module according to a first modification of the second embodiment; and

FIG. 13 is a partial cross-section for explaining a structure of a principal part of an ultrasonic transducer module according to a second modification of the second embodiment.

### DETAILED DESCRIPTION

Forms to implement the present disclosure (hereinafter, embodiments) are explained below with reference to the drawings. Embodiments explained below are not intended to limit the present disclosure. Furthermore, like reference symbols are assigned to like parts throughout the drawings.

### First Embodiment

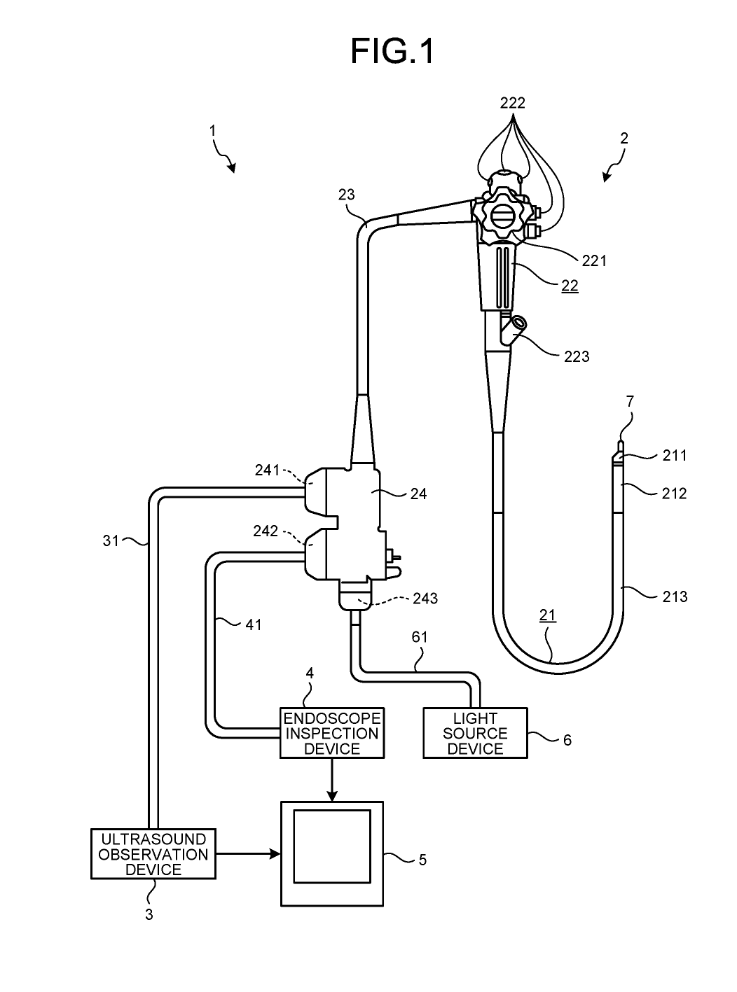

FIG. 1 is a diagram schematically showing an endoscope system according to a first embodiment. An endoscope system 1 is a system to perform ultrasonic diagnosis of the inside of a subject, such as a human, by using an ultrasonic endoscope. This endoscope system 1 includes, as shown in FIG. 1, an ultrasonic endoscope 2, an ultrasonic observation device 3, an endoscope inspection device 4, a display device 5, and a light source device 6.

The ultrasonic endoscope 2 irradiates, to a subject, an ultrasonic pulse (acoustic pulse) that is obtained by converting an electrical pulse signal received from the ultrasonic observation device 3 by an ultrasonic transducer that is provided at a distal end portion thereof, and outputs an electrical echo signal expressed by voltage change that is obtained by converting an ultrasonic echo reflected by the subject.

The ultrasonic endoscope 2 includes an imaging optical system and an imaging device, and is capable of imaging any one of a digestive canal and a respiratory organ, inserted into a digestive canal (the esophagus, the duodenum, the large intestine) or a respiratory organ (the trachea, the bronchus). Moreover, it is capable of imaging the peripheral organs (the pancreas, the gallbladder, the biliary duct, the biliary tract, a lymph node, a mediastinum organ, a blood vessel, and the like) by using ultrasonic waves. Furthermore, the ultrasonic endoscope 2 includes a light guide that guides illumination light to be irradiated to the subject an optical imaging. A distal end portion of this light guide reaches to a distal end of an insertion portion of the ultrasonic endoscope 2 inserted to a subject, and a proximal end portion thereof is connected to the light source device 6 that generates the illumination light.

The ultrasonic endoscope 2 includes, as shown in FIG. 1, an insertion portion 21, an operation portion 22, a universal cord 23, and a connector 24. The insertion portion 21 is a portion to be inserted into the subject. This insertion portion 21 includes, as shown in FIG. 1, a hard distal end portion 211 that is arranged at a distal end and that holds an ultrasonic transducer 7, a bending portion 212 that is connected to a proximal end side of the distal end portion 211 and that is bendable, and a flexible tube 213 that is connected to a proximal end side of the bending portion 212 and that has flexibility. Although a specific illustration is omitted, in the insertion portion 21, a light guide that transmits illumination light supplied by the light source device 6 and plural signal cables that transmit various kinds of signals are routed, and a treatment-tool insertion channel to insert a treatment tool is formed.

The ultrasonic transducer 7 is a convex ultrasonic transducer that is provided with multiple piezoelectric elements in an array, and that performs electronic scanning by electronically switching piezoelectric elements to be involved in transmission and reception, or by giving a delay time to transmission and reception of the respective piezoelectric elements. A structure of the ultrasonic transducer 7 is described later.

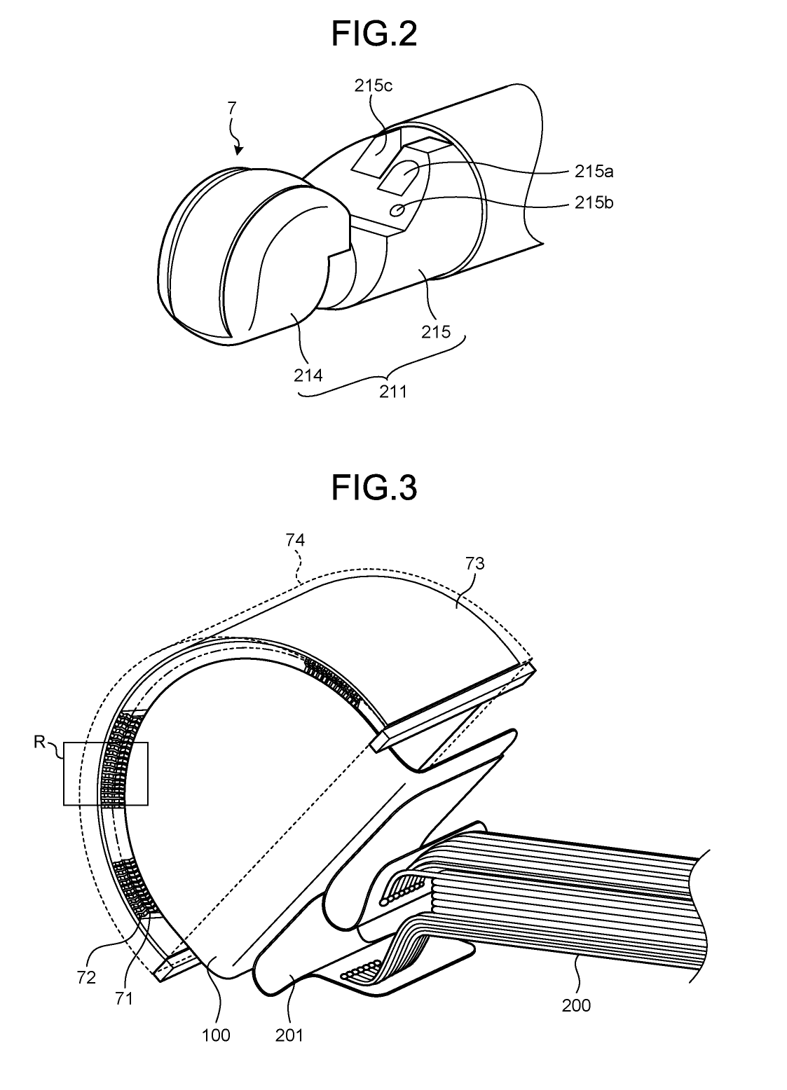

FIG. 2 is a perspective view schematically showing a distal end structure of the insertion portion of the ultrasonic endoscope according to the first embodiment. As shown in FIG. 2, the distal end portion 211 includes an ultrasonic transducer module 214 holding the ultrasonic transducer 7, and an endoscope module 215 having an objective lens 215a that constitutes a part of the imaging optical system, and through which light is taken in from the outside and an illumination lens 215b that condenses illumination light to emit it the outside. In the endoscope module 215, a treatment-tool protrusion outlet 215c that communicates with the treatment-tool insertion channel formed in the insertion portion 21 and that lets a treatment tool protrude out from a distal end of the insertion portion 21 is formed. The treatment-tool insertion channel is arranged such that a portion near an end communicating with the treatment-tool protrusion outlet 215c is tilted with respect to a longitudinal axis of the insertion portion 21, and that a treatment tool protrudes out in a direction tilted with respect to the longitudinal axis from the treatment-tool protrusion outlet 215c. The longitudinal axis herein is an axis along a direction of length of the insertion portion 21. While the axis direction varies in the bending portion 212 or the flexible tube 213 according to the position, the longitudinal axis in the hard distal end portion 211 is an axis of an invariant straight line.

The operation portion 22 is a portion that is connected to a proximal end side of the insertion portion 21, and that accepts various kinds of operation from a user, such as a doctor. This operation portion 22 includes, as shown in FIG. 1, a bending knob 221 to bend the bending portion 212, and plural operating members 222 to perform various kinds of operation. Moreover, in the operation portion 22, a treatment tool inlet 223 that communicates with the treatment-tool insertion channel, to insert a treatment tool into the treatment-tool insertion channel is formed.

The universal cord 23 is a cable that extends from the operation portion 22, and in which multiple signal cables to transmit various kinds of signals, an optical fiber to transmit illumination light supplied by the light source device 6, and the like are arranged.

The connector 24 is provided at a distal end of the universal cord 23. The connector 24 includes first to third connector portions 241 to 243 to which an ultrasonic cable 31, a video cable 41, and an optical fiber cable are connected, respectively.

The ultrasonic observation device 3 is electrically connected to the ultrasonic endoscope 2 through the ultrasonic cable 31 (refer to FIG. 1), and outputs a pulse signal to the ultrasonic endoscope 2 through the ultrasonic cable 31, and receives an echo signal input from the ultrasonic endoscope 2. The ultrasonic observation device 3 subjects the echo signal to a predetermined process to generate an ultrasonic image.

The endoscope inspection device 4 is electrically connected to the ultrasonic endoscope 2 through the video cable 41 (refer to FIG. 1), and receives an image signal input from the ultrasonic endoscope 2 through the video cable 41. The endoscope inspection device 4 subjects the image signal to a predetermined process to generate an endoscopic image.

The display device 5 is structured using a liquid crystal or an organic electroluminescence (EL), a projector, a cathode ray tube (CRT), or the like, and displays an ultrasonic image generated by the ultrasonic observation device 3, an endoscopic image generated by the endoscope inspection device 4, and the like.

The light source device 6 is connected to the ultrasonic endoscope 2 through an optical fiber cable 61 (refer to FIG. 1), and supplies illumination light to illuminate inside the subject to the ultrasonic endoscope 2 through the optical fiber cable 61.

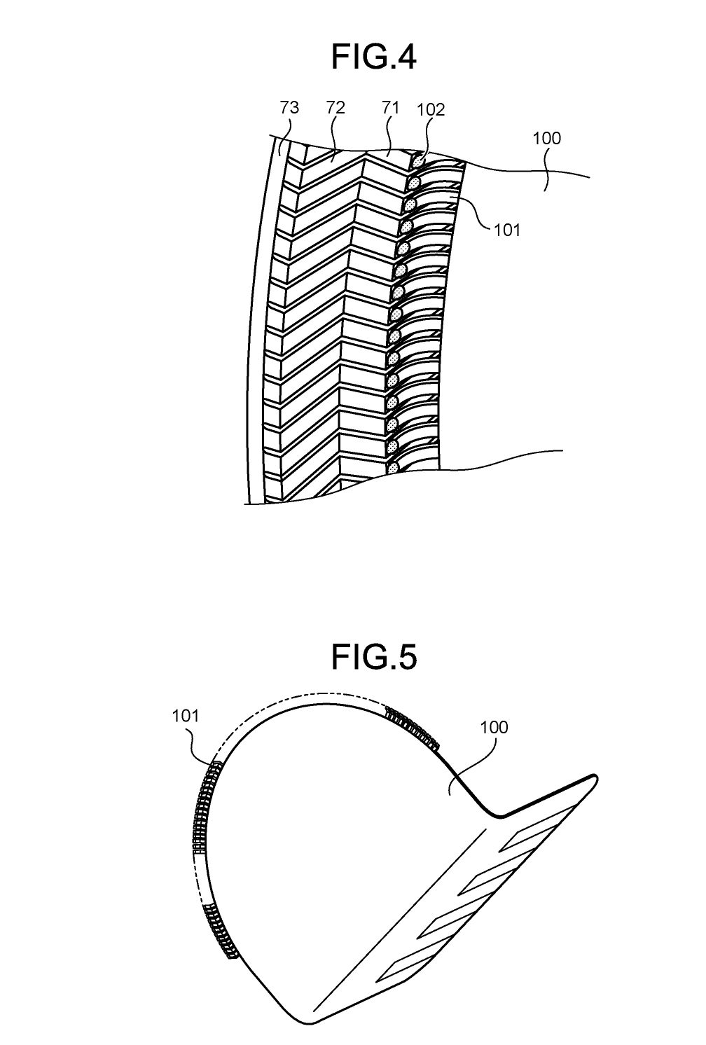

Subsequently, a structure of the ultrasonic transducer 7 arranged at a distal end of the insertion portion 21 is explained, referring to FIG. 2 to FIG. 5. FIG. 3 is a perspective view schematically showing a structure of the ultrasonic transducer module according to the first embodiment. FIG. 4 is a diagram for explaining a structure of a principal part of the ultrasonic transducer module shown in FIG. 3, and is a diagram showing a structure of a region R shown in FIG. 3. In the first embodiment, explanation is given assuming that the ultrasonic transducer 7 is a convex ultrasonic transducer as shown in FIG. 2, and that the piezoelectric elements 71 are of one-dimensional array (1D array) in which the piezoelectric elements 71 are arranged in a single row. In other words, in the ultrasonic transducer 7 according to the first embodiment, the piezoelectric elements 71 are arranged in a curved manner along an external surface of the ultrasonic transducer 7 forming a curved surface, and transmit and receive ultrasonic waves on a plane including a longitudinal axis and parallel to the longitudinal direction.

The ultrasonic transducer 7 includes plural piezoelectric elements 71 in a rectangular column shape arranged aligning the longitudinal axes, a first acoustic-matching layer 72 arranged on an external surface of the ultrasonic transducer 7 relative to the piezoelectric elements 71, a second acoustic-matching layer 73 that is arranged on the opposite side to a side of the first acoustic-matching layer 72 in contact with the piezoelectric elements 71, and an acoustic lens 74 that is arranged on the opposite side to a side of the second acoustic-matching layer 73 in contact with the first acoustic-matching layer 72. On the opposite side to a side of the piezoelectric elements 71 in contact with the first acoustic-matching layer 72, a backing material not shown is arranged. The backing material attenuates unnecessary ultrasonic vibrations that are caused by action of the piezoelectric elements 71. The backing material is made from a material having a large attenuation factor, for example, by using epoxy resin in which filers, such as alumina and zirconia, are dispersed, or rubber in which the above fillers are dispersed.

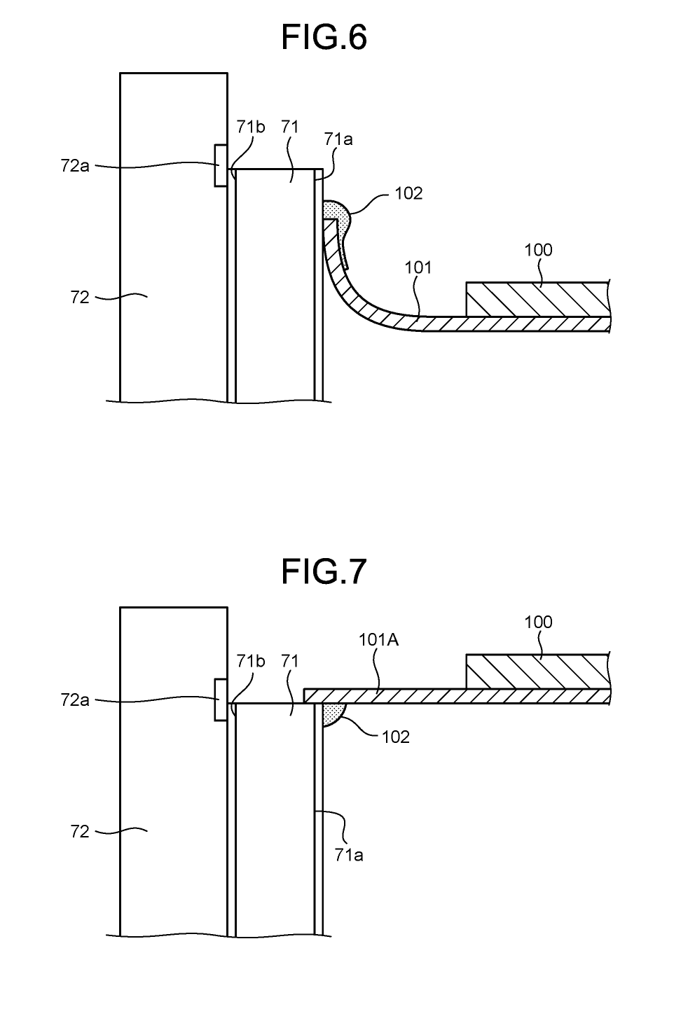

The piezoelectric element 71 converts an electrical pulse signal into an acoustic pulse and irradiate it to the subject, and converts an ultrasonic echo reflected by the subject into an electrical echo signal expressing the ultrasonic echo by voltage change to output. The piezoelectric element 71 has, for example, a signal input/output electrode 71a on a principal surface on a side facing the backing material, and a ground electrode 71b for grounding on a principal surface on the side of the piezoelectric element 71 facing the first acoustic-matching layer 72 (refer to FIG. 6). The respective electrodes are formed by using a metallic material or a resin material having conductivity. The principal surface herein signifies an acoustic radiation plane and a plane opposing to the acoustic radiation plane, and a plane intersecting the principal surface is referred to as side surface.

The first acoustic-matching layer 72 and the second acoustic-matching layer 73 match an acoustic impedance of the piezoelectric element 71 and an acoustic impedance to be observed for efficient transmission of sound (ultrasonic) between the piezoelectric element 71 and an object to be observed. The first acoustic-matching layer 72 and the second acoustic-matching layer 73 are made from materials different from each other. Although the first embodiment is explained supposing that two acoustic matching layers (the first acoustic-matching layer 72 and the second acoustic-matching layer 73) are provided, the matching layer can be one layer or three or more layers according to the characteristics of the piezoelectric element 71 and an object to be observed.

In the first acoustic-matching layer 72, a ground electrode 72a that is electrically connected to the ground electrode 71b of the piezoelectric element 71 is provided (refer to FIG. 6). The ground electrode 72a is made from a conductive material having larger impedance than the acoustic impedance of the piezoelectric element 71, and functions as a de-matching layer. The piezoelectric element 71 is grounded outside through the ground electrode 72a.

The acoustic lens 74 covers an external surface of the first acoustic-matching layer 72 and the second acoustic-matching layer 73. The acoustic lens 74 forms an external surface of the ultrasonic transducer 7. The acoustic lens 74 is formed by using silicone, polyethylpentene, epoxy resin, polyetherimide, or the like, has a convex or concave shape on one side to have a function of reducing ultrasonic waves, and emits ultrasonic waves that have passed through the second acoustic-matching layer 73 to the outside or takes in an ultrasonic echo from the outside. The acoustic lens 74 can be arranged arbitrarily, and a structure without the acoustic lens 74 is also applicable.

The ultrasonic transducer 7 having the above structure irradiates ultrasonic waves to an object to be observed through the first acoustic-matching layer 72, the second acoustic-matching layer 73, and the acoustic lens 74 by the piezoelectric elements 71 vibrating as a pulse signal is input thereto. At this time, in the piezoelectric element 71, the backing material attenuates unnecessary ultrasonic vibrations from the piezoelectric element 71 on the opposite side to a side on which the first acoustic-matching layer 72, the second acoustic-matching layer 73, and the acoustic lens 74 are arranged. Moreover, an ultrasonic wave reflected from the object to be observed is transmitted to the piezoelectric element 71 through the acoustic lens 74, the second acoustic-matching layer 73, and the first acoustic-matching layer 72. The piezoelectric element 71 vibrates by the transmitted ultrasonic wave, converts the vibration into an electrical echo signal, and outputs the echo signal to the ultrasonic observation device 3 through a wiring member 101 described later.

FIG. 5 is a schematic diagram showing a structure of a principal part of the ultrasonic transducer module according to the first embodiment, and is a schematic diagram for explaining a structure of a relay board. The ultrasonic transducer module 214 includes a relay board 100 that relays electrical connection between the ultrasonic transducer 7 and plural signal lines 200 (refer to FIG. 3) constituting a part of a path that electrically connects between the ultrasonic transducer 7 (the ultrasonic transducer module 214) and the ultrasonic observation device 3. The relay board 100 is a flexible board (flexible printed circuits (FPC)) that is held by the ultrasonic transducer 7 at a side portion of the ultrasonic transducer 7. The relay board 100 corresponds to a substrate in the present disclosure, and is electrically connected to the signal lines 200 through a second relay board 201. The relay board 100 is formed by arranging wirings on a substrate from polyimide. Furthermore, the relay board 100 is electrically connected to the signal input/output electrode 71a by wiring members 101 extending out from one surface of the relay board 100. In the first embodiment, the wiring members 101 extend out from one principal surface of the relay board 100. The relay board 100 has an arc shape along the alignment of the piezoelectric elements 71 at an edge on the side from which the wiring member 101 extend out.

FIG. 6 is a partial cross-section for explaining a structure of principal part of the ultrasonic transducer module shown in FIG. 3, and is a partial cross-section cut along a plane perpendicular to a direction of arrangement of the piezoelectric elements 71 and passing through the wiring members 101. The wiring member 101 is a flying lead that is formed by using a conductive material, such as copper, and an alloy, a principal component of which is copper. The wiring members 101 extend outside from part of the relay board 100, and has an L-shape formed by bending an end portion on a side connected to the signal input/output electrode 71a. To support formation of a joint portion 102, nickel/gold plating or solder plating can be formed on a surface of the wiring members 101.

Each of the wiring members 101 is joined with the signal input/output electrode 71a by the joint portion 102 at a bent distal end portion. The joint portion 102 is an electrolytic plating layer that is formed by an electrolytic plating method using a conductive material, such as nickel, copper, solder, and an alloy, a principal component of which is nickel, copper, or tin. The electrolytic plating method enables a quantitative control of material to form the joint portion 102 by controlling voltage and time. The joint portion 102 can be formed by joining by solder, or can be formed by molten soldering.

It is preferable that a joint portion formed by the joint portion 102 be a region piezoelectrically inactive for the piezoelectric element 17 to perform transmission and reception of ultrasonic waves accurately. Being piezoelectrically inactive is being not polarized, or being without application of an electric field.

Subsequently, a manufacturing method to manufacture the ultrasonic transducer module 214 described above is explained. First, the first acoustic-matching layer 72 and the second acoustic-matching layer 73 are laminated on the piezoelectric element 71. At this time, the ground electrode 71b in the piezoelectric element 71 and the ground electrode 72a in the first acoustic-matching layer 72 come into contact with each other.

Thereafter, the signal input/output electrode 71a and the wiring members 101 are joined by the joint portion 102 in a state in which the signal input/output electrode 71a of the piezoelectric elements 71 and the wiring members 101 are in contact with each other. The joint portion 102 is formed, for example, by using the electrolytic plating method described above. By using the electrolytic plating method, generation of heat at the time of joining the signal input/output electrode 71a and the wiring members 101 can be suppressed, and thereby enabling to suppress thermal deterioration of the piezoelectric elements 71. Moreover, by using the electrolytic plating method, multiple sets of the signal input/output electrode 71a and the wiring members 101 can be joined all together, thereby enabling to reduce manufacturing cost. The joint portion 102 can be formed by joining by solder or by molten soldering as described above.

The manufacturing procedure described above can be in reverse order. Specifically, after the signal input/output electrode 71a and the wiring members 101 are joined, the first acoustic-matching layer 72 and the second acoustic-matching layer 73 can be laminated on the piezoelectric element 71.

Thereafter, the backing material is filled on the opposite side to the first acoustic-matching layer 72 of the piezoelectric element 71, and the acoustic lens 74 is mounted. Furthermore, this acoustic lens 74 is placed in the casing. Thus, the ultrasonic transducer module 214 shown in FIG. 2 is manufactured.

According to the first embodiment explained above, when the piezoelectric elements 71 and the relay board 100 are joined, the signal input/output electrodes 71a of the piezoelectric elements 71 and the L-shaped wiring members 101 extending from the relay board 100 are joined by the joint portion 102. Because the wiring members 101 integrated with the relay board 100 are joined with the piezoelectric elements 71, respectively, the positioning accuracy in connecting the piezoelectric elements 71 and the relay board 100 can be improved compared to the case when independent multiple signal lines are connected to the piezoelectric elements 71 one by one. Thus, the array density of the piezoelectric elements 71 can be increased while a sufficient joint strength between the piezoelectric element 71 and the wiring member 101 is kept.

Furthermore, according to the first embodiment, the distal end of the wiring member 101 is bent in an L-shape to connect this distal end with the signal input/output electrode 71a and, therefore, stress externally applied thereto can be dispersed by this L-shape, and stress on the piezoelectric elements 71 can be reduced.

In the first embodiment described above, it has been explained that the relay board 100 is electrically connected to the signal lines 200 through the second relay board 201, but it can be directly connected to the signal lines 200 without the second relay board 201.

First Modification of First Embodiment

FIG. 7 is a partial cross-section for explaining a structure of a principal part of an ultrasonic transducer module according to a first modification of the first embodiment. As shown in FIG. 7, in the first modification, a wiring member 101A extending in a flat plate shape is included in addition to the wiring members 101 described above. As the first modification, wiring members that are not bent in an L-shape are also applicable, and the positioning accuracy in connecting the piezoelectric elements 71 and the relay board 100 can be improved compared to the case in which independent multiple signal lines are connected to the piezoelectric elements 71 one by one.

Second Modification of First Embodiment

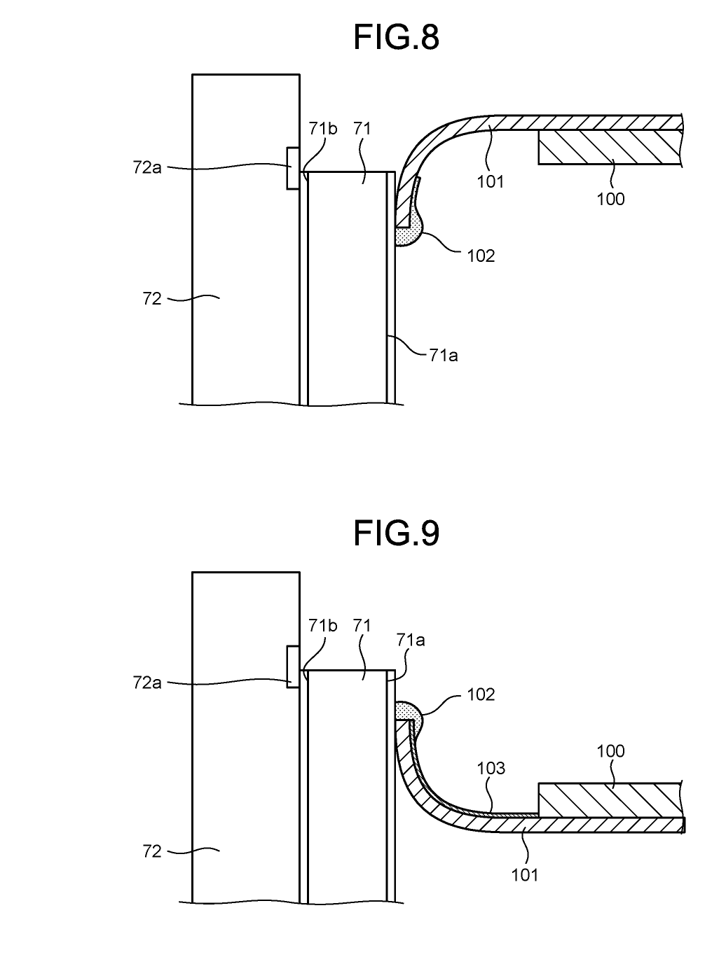

FIG. 8 is a partial cross-section for explaining a structure of a principal part of an ultrasonic transducer module according to a second modification of the first embodiment. As shown in FIG. 8, in the second modification, the wiring members 101 described above are positioned outside a region extending in a lamination direction of the piezoelectric elements 71, the first acoustic-matching layer 72, and the second acoustic-matching layer 73, that is, outside a region according to the matching layer, except the joint portion by the joint portion 102. By arranging the wiring members 101 outside the region according to the matching layer as the second modification, ultrasonic waves are not transmitted to the wiring members 101 from the piezoelectric elements 71, and ultrasonic waves reflected by the wiring members 101 do not enter the piezoelectric elements 71. Therefore, it is possible to suppress reception of unnecessary ultrasonic echo by the piezoelectric elements 71. Thus, suppressing noises due to an unnecessary ultrasonic echo, it is possible to enhance the image quality of ultrasonic images acquired by the ultrasonic transducer 7.

Moreover, other than the arrangement in the second modification described above, the wiring members 101 can be arranged in a region according to the de-matching layer formed by the ground electrode 72a. In this case also, ultrasonic waves reflected by the wiring members 101 do not enter the piezoelectric elements 71 as described above and, therefore, reception of an unnecessary ultrasonic echo by the piezoelectric elements 71 can be suppressed.

Third Modification of First Embodiment

FIG. 9 is a partial cross-section for explaining a structure of a principal part of an ultrasonic transducer module according to a third modification of the first embodiment. As shown in FIG. 9, in the third modification, an elastic deformable reinforcing layer 103 is arranged on a surface of the wiring members 101 where the relay board 100 is arranged, the surface being opposite to a surface where the signal input/output electrode 71a is connected.

The reinforcing layer 103 is, for example, made from polyimide, which is the same material forming the relay board 100. The reinforcing layer 103 extends up to the distal end of the wiring members 101 from an end portion of the relay board 100, and gives resilience to the wiring members 101. This enables to increase a load applied to the signal input/output electrode 71a by the wiring members 101 by the reinforcing layer 103 when the wiring members 101 are pressure-welded to the signal input/output electrode 71a, and to obtain firmer connection between the wiring members 101 and the signal input/output electrode 71a. Moreover, by arranging the reinforcing layer 103, it is possible to improve the shape stability of the wiring members 101, enabling to suppress a break of the wiring member 101 at an end portion on a side extending from the relay board 100, or to suppress deformation of the wiring members 101.

Note that as long as the reinforcing layer 103 is elastic and deformable and capable of giving resilience to wiring members, the material thereof is not limited to polyimide described above. Moreover, the thickness of the reinforcing layer 103 can be adjusted appropriately according to a load applied to the piezoelectric elements 71 by the wiring members 101, as long as it is equal to or smaller than the thickness of the relay board 100. Furthermore, by combining with the second modification described above, it is possible to obtain firmer connection between the wiring members 101 and the signal input/output electrode 71a, while suppressing reception of unnecessary ultrasonic echoes by the piezoelectric elements 71.

Moreover, the reinforcing layer 103 is not necessarily required to be arranged for all of the wiring members 101, but can be arranged for part of the wiring members 101. Furthermore, although it has been explained that the reinforcing layer 103 extends up to the distal end of the wiring members 101 from the end portion of the relay board 100, it can be arranged partially at part of that from the end of the relay board 100 to the distal end of the wiring members 101.

### Second Embodiment

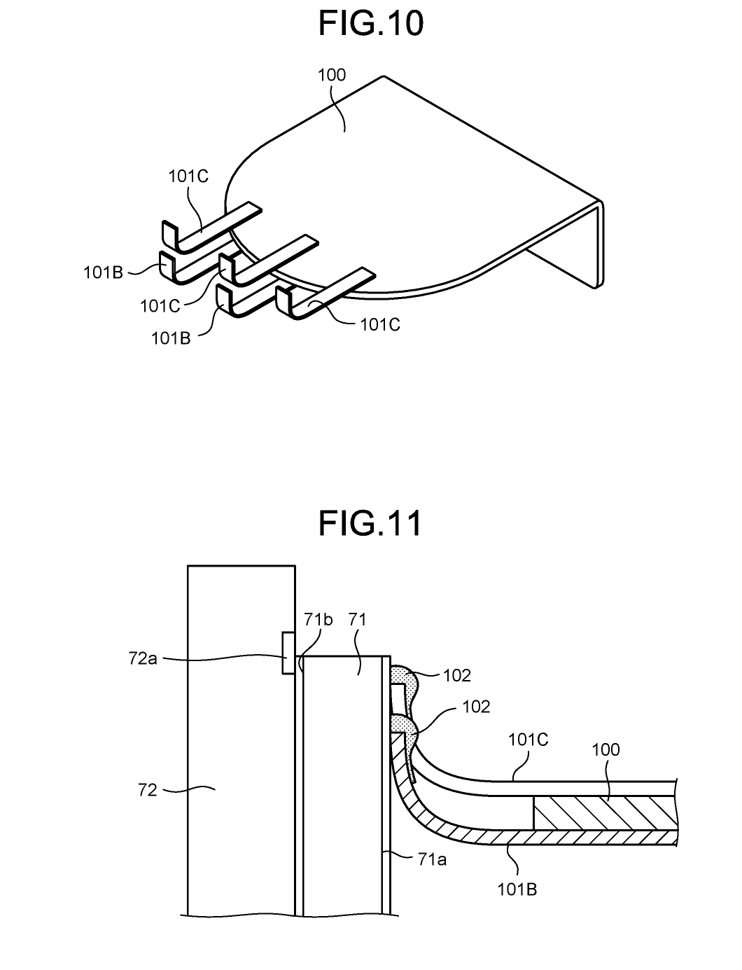

FIG. 10 is a schematic diagram showing a structure of a principal part of an ultrasonic transducer module according to a second embodiment, and is a schematic diagram for explaining a structure of a relay board. While it has been explained that the wiring members 101 extend from one surface of the relay board 100 in the first embodiment, wiring members 101B and 101C respectively extend out from surfaces opposing to each other of the relay board 100 in the second embodiment. FIG. 10 shows a structure in which five pieces of wiring members extend out for explanation, but the number of wiring members arranged in an actual situation depends on the number of the piezoelectric elements 71. The wiring members 101B and 101C are connected to wiring patterns formed on the relay board 100, respectively.

In the relay board 100 shown in FIG. 10, plural wiring members 101B and plural wiring members 101C are arranged. The wiring members 101B extend out from one surface of the relay board 100. On the other hand, the wiring members 101B extend out from a surface different from the surface from which the wiring members 101B extend. In the second embodiment, explanation is given assuming that the wiring members 101B and the wiring members 101C extend out from principal surfaces opposing to each other. Moreover, in the second embodiment, the wiring members 101B and the wiring members 101C are arranged in a staggered arrangement in a planer view from a direction perpendicular the principal surface of the relay board 100. The wiring members 101B and 101C are arranged in a zig zag alignment in a side view.

The wiring members 101B and 101C are flying leads that are formed by using a conductive material, such as copper, and an alloy, a principal component of which is copper. The wiring members 101B and 101C have an L-shape formed by bending an end portion on a side connected to the signal input/output electrode 71a. The wiring members 101B and 101C have the same shape, and are held by the relay board 100 such that the bending directions are the same.

FIG. 11 is a partial cross-section for explaining a structure of a principal part of the ultrasonic transducer module according to the second embodiment, and is a partial cross-section cut along a plane perpendicular to the alignment direction of the piezoelectric elements 71 and passing through the wiring members 101B. The wiring members 101B and 101C are joined with the signal input/output electrodes 71a at bent distal end portions.

According to the second embodiment explained above, similarly to the first embodiment described above, when the piezoelectric elements 71 and the relay board 100 are joined, the signal input/output electrodes 71a of the piezoelectric elements 71 and the L-shaped wiring members 101B and 101C extending from the relay board 100 are joined by the joint portion 102. Because the wiring members 101 integrated with the relay board 100 are joined with the piezoelectric elements 71, respectively, the positioning accuracy in connecting the piezoelectric elements 71 and the relay board 100 can be improved compared to the case when independent multiple signal lines are connected to the piezoelectric elements 71 one by one. Thus, the array density of the piezoelectric elements 71 can be increased while a sufficient joint strength between the piezoelectric element 71 and the wiring member 101 is kept.

Moreover, according to the second embodiment, the wiring members 101B and 101C extend out from the surfaces of the relay board 100 opposing to each other, respectively, and are arranged in a staggered arrangement in a planer view from a direction perpendicular to the principal surface of the relay board 100. Therefore, intervals of wiring patterns in the relay board 100 can be widened. Thus, crosstalk between the wiring patterns can be suppressed.

Although it has been explained that the wiring members 101B and 101C respectively extend from the surfaces of the relay board opposing to each other, and are arranged in a staggered arrangement in a planer view from a direction perpendicular to the principal surface of the relay board 100 in the second embodiment described above, a different arrangement from the staggered arrangement, such as an arrangement in which the wiring members 101B are next to each other in the planar view described above, can be applied as long as they extend from the surfaces of the relay board 100 opposing to each other.

Furthermore, although explanation has been given with the example in which the wiring members 101B and 101C extend from the two surfaces of the relay board 100 opposing to each other in the second embodiment described above, it is not limited thereto as long as they extend from surfaces different from each other. The surfaces different from each other herein are surfaces divided by a portion bent with a predetermined radius of curvature (for example, 90°) on a surface a substrate as a boundary. For example, it can be arranged such that wiring members extend from a side surface. In this case, the wiring members 101B and 101C can be arranged to extend from one of the principal surfaces opposing to each other and a side surface, to extend from the other principal surface and the side surface, or to extend from one principal surface, the other principal surface, and the side surface, respectively.

Although it has been explained that the wiring members 101B and 101C have the same shape and held by the relay board 100 such that the bending directions are the same in the second embodiment, it is not limited thereto as long as the wiring members 101B and 101C can be connected to the signal input/output electrodes 71a. For example, one having a different radius of curvature can be included, or the bending directions can vary. In following first modification and second modification, other examples of wiring members are explained.

First Modification of Second Embodiment

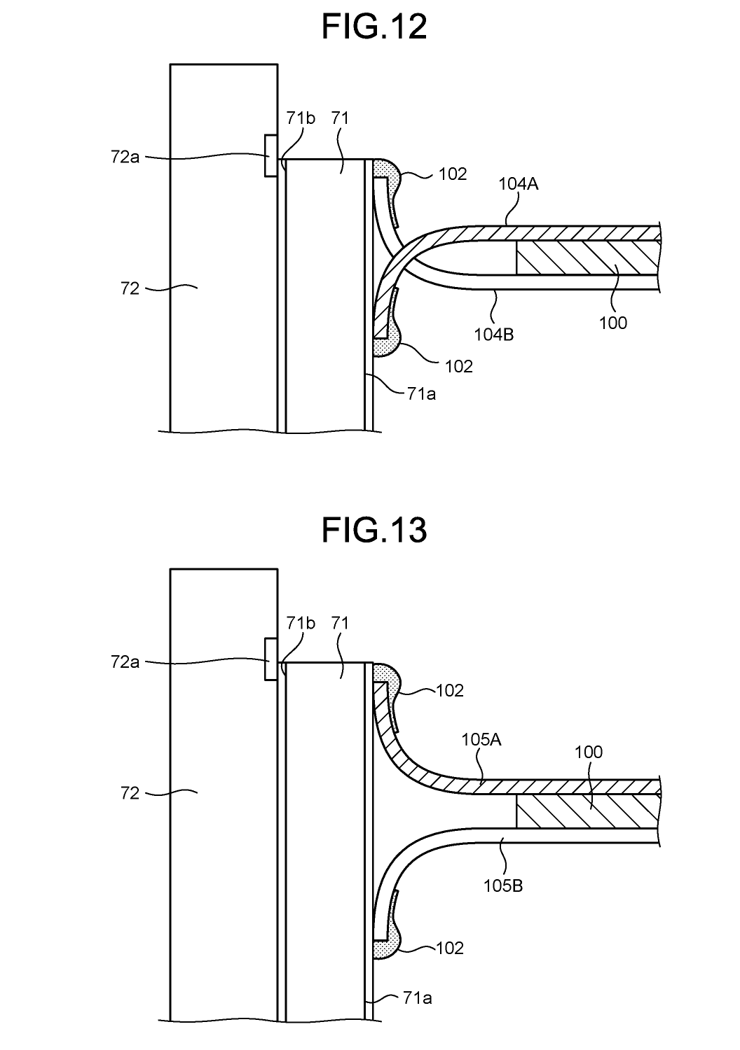

FIG. 12 is a partial cross-section for explaining a structure of a principal part of an ultrasonic transducer module according to a first modification of the second embodiment. As shown in FIG. 12, in the first modification, wiring members 104A, 104B are arranged such that bent portions that are joined by the joint portion 102 intersect with each other. When the wiring members 104A, 104B are arranged such that the bent portions intersect with each other as in this first modification, joint portions of the wiring members 104A, 104B in the piezoelectric elements 71 are separated compared to the case of the second embodiment described above. Therefore, positions at which loads are applied to the piezoelectric elements 71 by the wiring members 104A, 104B are dispersed, and concentration of the loads applied by the wiring members 104A, 104B to the piezoelectric elements 71 can be suppressed. Moreover, by separating the joint portions, operation of joining of wiring members 104A, 104B and the piezoelectric elements 71 can be facilitated.

Second Modification of Second Embodiment

FIG. 13 is a partial cross-section for explaining a principal part of an ultrasonic transducer module according to a second modification of the second embodiment. As shown in FIG. 13, in this second modification, wiring members 105A, 105B are arranged such that end portions joined by the joint portion 102 are separated from each other. When the wiring members 105A, 105B are arranged such that the end portions are separated from each other as in this second modification, the joint portions of the wiring members 105A, 105B in the piezoelectric elements 71 are further separated compared to the first modification of the second embodiment. Therefore, positions at which loads are applied to the piezoelectric elements 71 by the wiring members 105A, 105B are dispersed, and concentration of the loads applied by the wiring members 105A, 105B to the piezoelectric elements 71 can be further suppressed.

The embodiments to implement the present disclosure have been explained, but the present disclosure is not to be limited to the embodiments and the modifications described above. The present disclosure can include various embodiments within a range not departing from the technical ideas described in claims, not limited to the embodiments and the modifications explained above. Moreover, configurations of the embodiments and the modifications can be combined as appropriate.

Furthermore, although the above first and second embodiments have been explained using the piezoelectric elements as an example of a device that emits ultrasonic waves and converts ultrasonic waves entering from outside into an echo signal, it is not limited thereto, and it can be a device that is manufactured by using micro electro mechanical systems (MEMS), such as a capacitive micromachined ultrasonic transducer (C-MUT) and a piezoelectric micromachined ultrasonic transducer (P-MUT).

Moreover, as for the ultrasonic endoscope, it can be applied to a thin ultrasonic probe that performs scanning by mechanically rotating transducers without an optical system. The ultrasonic probe is usually inserted into the biliary tract, the biliary duct, the pancreatic duct, the trachea, the bronchus, the urethra, or the ureter, and used to observe the peripheral organs (the pancreas, the lungs, the prostate, the bladder, a lymph node, and the like).

Furthermore, the ultrasonic transducer can be any of a linear transducer, a radial transducer, and a convex transducer. When the ultrasonic transducer is a linear transducer, the scanning region is in a rectangular shape (rectangle, square), and the ultrasonic transducer is a radial transducer or a convex transducer, the scanning region is in a fan shape or in an annular shape. Moreover, the ultrasonic endoscope can be one that causes ultrasonic transducers to scan mechanically, or one in which multiple devices as ultrasonic transducers are arranged in an array, and in which devices to be involved in transmission and reception are electronically switched, or delay time is given to transmission and reception of the respective devices, to perform scanning electronically.

Moreover, as the ultrasonic endoscope, an external ultrasonic probe that irradiates ultrasonic waves from a body surface of a subject can be applied. The external ultrasonic probe is usually used to observe abdominal organs (the liver, the gallbladder, the bladder), the breast (particularly, mammary glands), and the thyroid.

According to the present disclosure, an effect that an array density of piezoelectric elements can be increased while a sufficient joint strength between a piezoelectric element and a wiring is kept is obtained.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic transducer module comprising:
piezoelectric elements, each being aligned in the same direction that is a longitudinal direction thereof;
electrodes that are formed on surfaces of the respective piezoelectric elements;
a substrate that includes wiring members extending from at least one surface of the substrate, the wiring members being connected one by one to the electrodes of the piezoelectric elements; and
reinforcing layers that are provided on a surface of each of the wiring members, the surface being opposite to a surface of the corresponding wiring member where the corresponding electrode is connected.

2. The ultrasonic transducer module according to claim 1, wherein each of the wiring members is bent at an end portion connected to the corresponding electrode.

3. The ultrasonic transducer module according to claim 1, wherein each of the reinforcing layers is formed with a material that is the same as a material of the substrate.

4. The ultrasonic transducer module according to claim 1, wherein the wiring members extend from different surfaces of the substrate.

5. The ultrasonic transducer module according to claim 3, wherein the wiring members are arranged in a staggered arrangement in a planer view from a direction perpendicular to the principal surface of the substrate.

6. The ultrasonic transducer module according to claim 1, wherein the piezoelectric elements are arranged along a curved surface.

7. An ultrasonic endoscope comprising:
an ultrasonic transducer module that is provided at a distal end portion of the ultrasonic endoscope, the ultrasonic transducer module including
piezoelectric elements, each being aligned in the same direction that is a longitudinal direction thereof,
electrodes that are formed on surfaces of the respective piezoelectric elements,
a substrate that includes wiring members extending from at least one surface of the substrate, the wiring members being connected one by one to the electrodes of the piezoelectric elements, and
reinforcing layers that are provided on a surface of each of the wiring members, the surface being opposite to a surface of the corresponding wiring member where the corresponding electrode is connected; and
an insertion portion that is inserted into a subject.
